(19) United States
(12) Patent Application Publication
(10) Pub. No.: US 2024/0293429 A1
(43) Pub. Date: Sep. 5, 2024

(54) MICROBIAL-TRIGGERED ORAL INTESTINAL DRUG DELIVERY FORMULATION AND METHOD OF PREPARATION THEREOF

(71) Applicant: NBI BIOSCIENCES PVT LTD, Gurugram, Haryana (IN)

(72) Inventors: ATUL KUMAR, Gurugram, Haryana (IN); Braj Gaurav SHARMA, Baldeo, Mathura (IN)

(21) Appl. No.: 18/575,988

(22) PCT Filed: May 30, 2022
(86) PCT No.: PCT/IN2022/050499

§ 371 Date: Jan. 2, 2024

(30) Foreign Application Priority Data
Jun. 1, 2021 (IN) 202121024439

(51) Int. Cl. A61K 31/58; A61K 9/20; A61K 9/28

(52) U.S. Cl. CPC A61K 31/58; A61K 9/2013; A61K 9/2054; A61K 9/286; A61K 9/2866; A61K 9/2893

(57) ABSTRACT

The present disclosure relates to microbial-triggered small and large intestinal drug delivery (SLIDD) oral formulation and method of preparation thereof. The formulation delivers the active ingredient or the drug to the predetermined location in the GI tract with success.

### FIELD OF THE INVENTION

The invention relates to microbial-triggered small and large intestinal drug delivery (SLIDD) oral formulation and method of preparation thereof.

### BACKGROUND OF THE INVENTION

Intravenous (IV), intramuscular (IM), intranasal (IN), intradermal (ID)/transdermal and oral administration are the main routes by which drug/s are delivered. The choice of appropriate delivery route is dependent upon the absorption mechanism as well as the nature of the drug for achieving the highest bioavailability and effectivity. Conventional alternative drug delivery methods such as intravenous and intramuscular delivery have several drawbacks including pain and risk of infection, requirements for the use of sterile techniques and the associated risks of maintaining an IV line in a patient for an extended period of time. However, amongst the various drug delivery routes, the most preferred and common route of drug administration is the oral route, primarily due to ease of use, non-invasive process, and convenience of self-administration. This route is preferred for delivering drugs in the gastrointestinal tract (GI tract) for both systemic drug delivery and for treating local gastrointestinal diseases.

The intestine is believed to be a suitable absorption site for drugs generally because of the abundant presence of the digestive enzymes, presence of intestinal mucosa and enzymatic degradation that releases the drug into ileum or colon which leads to greater systemic bioavailability. Moreover, coupled with the long residence time of the intestine, the intestinal drug delivery is preferred as a portal for the entry of drugs into the systemic circulation.

Despite the clear advantages, oral drug delivery targeted to the intestine can be challenging due to the harsh conditions of the human gastrointestinal tract. Apart from the harsh conditions, the GI tract also displays several physiological barriers that affect drug delivery, thereby degrading/denaturing the drug compounds, causing gastric irritation and poor or slow absorption of the drug.

There are different approaches for delivering the drugs to the intestines; most common are the pH dependent approach. These pH dependent systems allow the drugs to bypass the acidic pH of the stomach and release the drug into the intestine. However, the peristaltic movement of intestine pushes the pH-based formulation from the small intestine to the excretion pathway. Since the drug is retained for a shorter time in the small intestine; the dosage of the drug is increased to negate the shorter retention time. Apart from drug wastage, this approach also leads to certain side-effects. Moreover, the pH varies individually thereby making this delivery to the desired location fraught with uncertainty. Further, this approach is not suitable for sensitive drugs wherein increase in the dosage or delayed release of the drug due to any imbalance in the pH may might lead to serious complications.

Thus, there is a need for an improved oral formulation for delivering a precise amount of a pharmaceutical product to the small intestine and colon, without premature delivery of the product to the upper gastrointestinal (GI) tract and also ameliorating at least one of the aforementioned drawbacks.

### SUMMARY OF THE INVENTION

In one aspect, the present invention provides an oral drug delivery formulation comprising:
a central core;
an inner coating of the central core; and
an optional outer enteric coating
wherein the central core comprises an active ingredient or drug having a primary coating based on plant extract or their derivatives and one or more disintegration agent along with one or more pharmaceutically acceptable excipients.

In another aspect, the present invention provides a method for preparing the oral drug delivery formulation.

In still another aspect, the present invention provides a method for the treatment of a patient by delivering the drug to a predetermined location in the GI tract, preferably colon.

In yet another aspect, the present invention relates to use of the oral drug delivery formulation for delivering a drug to a predetermined location in the gastrointestinal tract, preferably colon

### BRIEF DESCRIPTION OF THE DRAWINGS

Reference will be made to embodiments of the invention, examples of which may be illustrated in accompanying figures. These figures are intended to be illustrative, not limiting. Although the invention is generally described in the context of these embodiments, it should be understood that it is not intended to limit the scope of the invention to these particular embodiments.

FIG. 1 shows the HPLC calibration curve of Budesonide (BDS)

FIG. 2 shows the percent cumulative drug release with respect to time.

FIG. 3 shows unidentified peaks of interaction of drug with tween 80.

FIG. 4 shows drug release studies by USP Type 1 apparatus in 10% ethanol in 1000 ml phosphate buffer pH 7.4 at 100 rpm.

FIG. 5 shows matrix integrity of NIP-BDS-13 to NIP-BDS-14 after 8 h and 24 h

FIG. 6 shows sequential dissolution studies of uncoated BDS-NIP-014 (Prototype-I) and coated BDS-NIP-014 (Prototype-II); Sequential dissolution by HPLC—1 h in 0.1N HCL, 2.5 h in pH 6 phosphate buffer with 10% ethanol and up to 30 h in pH 6.8 phosphate buffer with 10% ethanol.

FIG. 7 shows matrix integrity images of uncoated BDS-NIP-014 (Prototype-I) and coated BDS-NIP-014 (Prototype-II) after 1 h in 0.1N HCl and 30 h in pH 6.8 phosphate buffer with 10 percent ethanol; (a) Coated 1 h tablet in 0.1 HCl; (b) Uncoated 1 h tablet in 0.1 HCl; (c) Coated 30 h tablet in pH 6.8 phosphate buffer; (d) Coated 30 h tablet in pH 6.8 phosphate buffer.

FIG. 8 shows matrix integrity of uncoated BDS-NIP-014 (Prototype-I) and coated BDS-NIP-014 (Prototype-II) with and without cecal content; (a) Uncoated NIP-BDS-014 after 24 h without cecal content; (b) Coated NIP-BDS-014 after 24 h with 4% cecal content; (c) Coated NIP-BDS-014 after 24 h without cecal content.

FIG. 9 shows SPECT-Gamma camera image of subject NBI02 indicating the location of drug inside the GI tract at Ileo-cecal junction, Ascending, Transverse and Descending colon at 24 hours.

FIG. 10 shows the release pattern of drug in the GI tract. (A)—The drug in the stomach & small intestine and tablet remains intact without drug release (B)—Drug showed release at the ileocaecal junction/arrival of colon and drug release started (C)—Drug distributed in the colon and complete disintegration of tablet monitor.

### DETAILED DESCRIPTION OF THE INVENTION

The present invention discloses a microbial-triggered oral intestinal drug delivery formulation. The human intestine harbors higher microbial flora, which has been targeted in developing the microbial-triggered oral intestinal drug delivery formulation of the present invention. The formulation of the present invention delivers the drug to a predetermined location in the GI tract.

The formulation of the present invention comprises at least three parts; outer enteric coating, an inner coating and central core. These three parts of this formulation function to provide for release of drug to the colon without premature delivery of drug to the upper GI tract.

Accordingly, the present invention is directed towards an oral drug delivery formulation comprising:
a central core;
an inner coating of the central core; and
an optional outer enteric coating
wherein the central core comprises an active ingredient or drug having a primary coating based on plant extract or their derivatives and one or more disintegration agent along with one or more pharmaceutically acceptable excipients.

As used herein, the phrase “active ingredient or drug” refers to those compounds or materials which function as an active pharmaceutical ingredient (API) for veterinary use as well as human pharmaceutical use.

As used herein, the phrase “Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

The formulation of the disclosure is formulated to be compatible with its intended oral route of administration. Accordingly, the excipient can be any excipient known in the art. In a preferred embodiment, the excipient is selected from the list consisting of, but not limited to, Magnesium Stearate, Talc, Silicone dioxide, Stearic acid, Sodium starch glycolate, Gelatin, Corn starch, Polyethylene glycol, and Starch.

The central core comprises an active ingredient/drug coated with a primary coating. The primary coating comprises at least one of plant extract, galactomannan, polysaccharide, chitosan, gum derived from plants such as guar gum and a disintegration agent such as micro crystalline cellulose (MCC), cross linked PVP, Cellulose, PVP, starch, alginic acid and calcium silicates. In an embodiment of the present invention the plant extract is selected from the group consisting of, but not limited to, Trigonella foenum-graecum, Cyamopsis tetragonoloba, tragacanth, Astragalus gummifer, Acasia senegal, acacia and nilotica plant belongs to family Fabaceae. In another embodiment of the present invention the galactomannan is selected from the group consisting of, but not limited to, guar gum, Trigonella foenum, gum acacia, gum arabic, gum tragacanth, gum karraya, gum ghatt, soya extract and corn extract. In still another embodiment of the present invention the polysaccharide is selected from the group consisting of, but not limited to, alginic acid, Chitosan, Pectin and derivatives.

In a preferred embodiment, the inner coating includes at least one polymer derived from acrylic acid, alginates, polysaccharides, and cellulose, preferably selected from a methacrylic acid polymer or pullulan.

In a preferred embodiment, the inner coating is preferably selected form hydroxypropyl methylcellulose (HPMC).

In a preferred embodiment, the inner coating further comprises at least one plasticizer coating. This coating prevents the exposure of the central core by providing the right amount of film flexibility to avoid cracking and peel-off. In an embodiment of the present invention, at least one plasticizer is selected from the group of, but not limited to, phthalates such as Di-isononyl phthalate (DINP), Di-isodecyl phthalate (DIDP), Di-2-ethyl hexyl phthalate (DEHP), and Di-n-octyl phthalate (DOP), and other such as Acetyl tri-butyl citrate (ATBC), Di(2-ethylhexyl) adipate (DEHA), Di-isononyl adipate (DINA), Di-isononyl-1,2-cyclohexanedicarboxylate (DINCH) and Alkyl sulphonic acid ester of Phenol (ASE).

The outer enteric coating is a polymer barrier that prevents its dissolution or disintegration in the gastric environment, i.e. it is resistant to gastric juices. This polymer barrier is fermented by the intestinal microbes/bacteria thereby releasing the drug in the large intestine. Further, the outer enteric coating has non-sticky property which supports the formulation for with enhanced transit inside the GI tract. The outer coating comprises at least one of semisynthetic polymer, anionic copolymer, plant extracts and plant derived resins and gum. In an embodiment of the present invention the semisynthetic polymer is selected from the group consisting of, but not limited to, hydroxypropyl methylcellulose (HPMC), polymer derived from acrylic acid, methacrylic acid, alginates, pullulan, and cellulose such as Diethyl phthalate triacetin. In another embodiment of the present invention the anionic copolymer is selected from the group consisting of, but not limited to, Eudragit-L-100, Eudragit S-100, HPMC, Methyl acrylate-methacrylic acid copolymers, Cellulose acetate phthalate (CAP), Cellulose acetate succinate, Hydroxypropyl methyl cellulose phthalate, Hydroxypropyl methyl cellulose acetate succinate, Polyvinyl acetate phthalate (PVAP), Methyl methacrylate-methacrylic acid copolymers, Shellac, Cellulose acetate trimellitate, Sodium alginate, corn starch (zein) ethyl cellulose, medium chain triglycerides, oleic acid, sodium alginate, stearic acid and Chitosan derivatives.

In yet another embodiment of the present invention the plant extract and derivatives are selected from the group consisting of, but not limited to, Trigonella foenum-graecum, Cyamopsis tetragonoloba, tragacanth, Astragalus gummifer, Acasia senegal, Acacia nilotica and plant belonging to the family Fabaceae also known as Leguminosae.

In a preferred embodiment, the plant extracts or their derivatives are selected from galactomannan, polysaccharide, chitosan, gum derived from plants.

In yet another preferred embodiment, the plant extract or a derivative thereof is based on guar gum.

In a preferred embodiment, the disintegration agent is selected from a micro crystalline cellulose, cross linked PVP, cellulose, PVP, starch, alginic acid and calcium silicates.

In still another embodiment, the inner coating further comprises at least one plasticizer, preferably selected from diethyl phthalate triacetin.

In a preferred embodiment, the formulation comprise an active ingredient or drug selected from the group consisting of, but not limited to, Budesonide, Methotrexate, Dexamethasone, Mesalamine, Metronidazole, Fidaxomicin, Capecitabine, 5-Fluorouracil, Sulphasalazine, Balsalazide, Mebeverine, Diclomine, Methylcellulose, Psyllium, Prednisone, Doxycycline, Metronidazole, Mercaptopurine, Cyclosporine, Mesalamine, Hydrocortisone, Prednisolone, 5-Flourouracil, Irinotecan Hydrochloride, Capecitabine, Methotrexate, paracetamol, dexamethasone, Fidaxomicin, amoxicillin, cefixime, tinidazole, metronidazole, Fluconazole, Oxaliplatin, Lubiprostone, Rifaximin, Alosetron etc.

In a preferred embodiment, the amount of outer coating is in the range of 1 to 50 wt % and inner primary coating is in the range of 1 to 25 wt % based on the total wt % of the formulation.

In a preferred embodiment, the amount of outer coating is in the range of 1 to 15 wt % and inner primary coating is in the range of 1 to 15 wt % based on the total wt % of the formulation.

In yet another preferred embodiment, the total excipient of the formulation is in the range of 1 to 50 wt % based on the total wt % of the formulation.

In yet another preferred embodiment, the total disintegration agent of the formulation is in the range of 1 to 30 wt % based on the total wt % of the formulation.

In yet another preferred embodiment, the total plant extracts or their derivatives are in the range of 1 to 50 wt % based on the total wt % of the formulation.

In another aspect, the present invention is directed towards a method for preparing an oral drug delivery formulation comprising the following steps:
primary coating of active ingredient or drug powder followed by drying and sifting to obtain a first mix;
granulating the first mix to obtain semi-dried granules;
milling the semi-dried granules followed by sifting to obtain sift milled granules;
predetermined amounts of disintegration agent along with one or more pharmaceutically acceptable excipients are mixed together and sieved to obtain a second mix;
blending the sift milled granules and the second mix to obtain the central core of formulation;
coating the central core to obtain the oral drug delivery formulation.

In a preferred embodiment of the method, the coating includes at least one polymer derived from acrylic acid, alginates, polysaccharides, and cellulose, preferably selected from a methacrylic acid polymer or pullulan, preferably comprises hydroxypropyl methylcellulose (HPMC).

In a preferred embodiment of the method, the disintegration agent is selected from a micro crystalline cellulose, cross linked PVP, cellulose, PVP, starch, alginic acid and calcium silicates.

In a preferred embodiment of the method, the blending is followed by a direct compression in tablet punching machine to obtain a tablet. In yet another preferred embodiment, the tablet is further outer enteric coated, preferably comprising Eudragit-L-100 or Eudragit S-100 or a combination thereof.

In a preferred embodiment, the method for preparing an oral drug delivery formulation comprises the following steps:
sifting separately, in pre-determined amounts, the active ingredient or drug powder, plant extract, galactomannan, polysaccharide, chitosan and a disintegration agent followed by blending for a pre-determined time period to obtain a first mix;
granulating the first mix along with the primary coat followed by drying to obtain semi-dried granules;
wet milling the semi-dried granules, drying, sifting followed by milling to obtain sift milled granules;
sifting the sift milled granules along with inner coating ingredients followed by blending to obtain a second mix; and
tableting the second mix followed by outer enteric coating to obtain the microbial-triggered oral large intestinal drug delivery formulation.

In a preferred embodiment of the method, the sifting is carried out in sieves having mesh size in the range of 30 to 400.

In a preferred embodiment of the method, the method comprises at least one step of wet granulation, direct compression, compressed coat and wet coating.

The drying is carried out considering the stability of the active ingredient or drug. In a preferred embodiment of the method, the preferred temperature range for drying is 25 to 65° C.

In yet another aspect, the present invention is related to a method for the treatment of a patient by delivering the drug to a predetermined location in the GI tract, comprising administering the formulation as claimed in claim 1 to a patient in need of the said drug.

The predetermined location in the GI tract is preferably colon and the drug is Budesonide.

In yet another aspect, the present invention is related to use of the oral drug delivery formulation for delivering active ingredient or drug to a predetermined location in the gastrointestinal tract, preferably ileocecal junction and colon.

The formulation of the present invention delivers the active ingredient or the drug to the predetermined location in the GI tract with more than 90%, preferably more than 95%, and most preferably more than 99% success.

### EXAMPLE

The following examples are illustrative of the invention but not limitative of the scope thereof:

### Materials and Methods

### Materials

Budesonide (BDS) was obtained from TCI Japan, lot number LP7RJ-SQ. Different HPMC grades were procured from Dow Chemicals. Avicel PH 101 was procured from Sigma Aldrich. Solvents like Acetonitrile and methanol were of HPLC grade while other solvents were of analytical grades.

### Method for the Preparation of the Formulation

Example 1 (NIP-BDS-01): 9 mg of Budesonide was mixed and coated with 50 mg guar gum. Further, 0.5 mg of Aerosil, 0.5 mg of magnesium stearate, and 40 mg of MCC PH101 were mixed together. Both the individual blends were sieved through 40 number mesh size. The two individual blends were blended together for 30 minutes at 100 RPM. Final blend underwent direct compression in tablet punching machine. The hardness of the tablet was set to 4.9 kPa. The final weight of the core tablet is 100 mg.

Example 2 (NIP-BDS-02): 9 mg of Budesonide was mixed and coated with 50 mg guar gum. Further, 0.5 mg of Aerosil, 0.5 mg of magnesium stearate, and 40 mg of MCC PH101 were mixed together. Both the individual blends were sieved through 40 number mesh size. The two individual blends were blended together for 30 minutes at 100 RPM. Final blend underwent direct compression in tablet punching machine. The hardness of the tablet was set to 9 kPa. The final weight of the core tablet is 150 mg. The coating solution was prepared using 50 mg of HPMC K100M. The coating was 6 wt %.

Example 3 (NIP-BDS-03): 9 mg of Budesonide was mixed and coated with 10 mg guar gum. Further, 0.5 mg of Aerosil, 0.5 mg of magnesium stearate, and 50 mg of MCC PH101 were mixed together Both the individual blends were sieved through 40 number mesh size. The two individual blends were blended together for 30 minutes at 100 RPM. Final blend underwent direct compression in tablet punching machine. The hardness of the tablet was set to 9 kPa. The final weight of the core tablet is 100 mg. The coating solution was prepared using 30 mg of HPMC E5. The coating was 6 wt %.

Example 4 (NIP-BDS-04): 9 mg of Budesonide was mixed and coated with 25 mg guar gum. Further, 0.5 mg of Aerosil, 0.5 mg of magnesium stearate, and 35 mg of MCC PH101 were mixed together. Both the individual blends were sieved through 40 number mesh size. The two individual blends were blended together for 30 minutes at 100 RPM. Final blend underwent direct compression in tablet punching machine. The hardness of the tablet was set to 9 kPa. The final weight of the core tablet is 100 mg. The coating solution was prepared using 30 mg of HPMC E5. The coating was 6 wt %.

Example 5 (NIP-BDS-05): 9 mg of Budesonide was mixed and coated with 30 mg guar gum. Further, 0.5 mg of Aerosil, 0.5 mg of magnesium stearate, and 40 mg of MCC PH101 were mixed together. Both the individual blends were sieved through 40 number mesh size. The two individual blends were blended for 30 minutes at 100 RPM. Final blend underwent direct compression in tablet punching machine. The hardness of the tablet was set to 9 kPa. The final weight of the core tablet is 100 mg. The coating solution was prepared using 20 mg of HPMC K4M. The coating was 6 wt %.

Example 6 (NIP-BDS-06): 9 mg of Budesonide was mixed and coated with 30 mg guar gum. Further, 0.5 mg of Aerosil, 0.5 mg of magnesium stearate, and 40 mg of MCC PH101 were mixed together. Both the individual blends were sieved through 40 number mesh size. The two individual blends were blended for 30 minutes at 100 RPM. Final blend underwent direct compression in tablet punching machine. The hardness of the tablet was set to 9 kPa. The final weight of the core tablet is 100 mg. The coating solution was prepared using 20 mg of HPMC K15M. The coating was 6 wt %.

Example 7 (NIP-BDS-07): 9 mg of Budesonide was mixed and coated with 30 mg guar gum. Further, 0.5 mg of Aerosil, 0.5 mg of magnesium stearate, and 40 mg of MCC PH101 were mixed together. Both the individual blends were sieved through 40 number mesh size. The two individual blends were blended for 30 minutes at 100 RPM. Final blend underwent direct compression in tablet punching machine. The hardness of the tablet was set to 9 kPa. The final weight of the core tablet is 100 mg. The coating solution was prepared using 20 mg of HPMC K100LVCR. The coating was 6 wt %.

Example 8 (NIP-BDS-08): 9 mg of Budesonide was mixed and coated with 30 mg guar gum. Further, 0.5 mg of Aerosil, 0.5 mg of magnesium stearate, and 40 mg of MCC PH101 were mixed together. Both the individual blends were sieved through 40 number mesh size. The two individual blends were blended for 30 minutes at 100 RPM. Final blend underwent direct compression in tablet punching machine. The hardness of the tablet was set to 9 kPa. The final weight of the core tablet is 100 mg. The coating solution was prepared using 20 mg of HPMC K100M. The coating was 6 wt %.

Example 9 (NIP-BDS-13): 0.5 mg of Aerosil, 0.5 mg of magnesium stearate, and 40 mg of Avicel PH101, were mixed together with budesonide 9 mg. Individual blends were sieved through 40 number mesh size. The two individual blends were blended for 30 minutes at 100 RPM. Final blend underwent direct compression in tablet punching machine. The hardness of the tablet was set to 9 kPa. The final weight of the core tablet is 150 mg. The coating solution was prepared using 20 mg of HPMC K100M. The coating was 6 wt %.

Example 10 (NIP-BDS-14): 9 mg of Budesonide was mixed and coated with 20 mg guar gum. Further, 0.5 mg of Aerosil, 0.5 mg of magnesium stearate, and 40 mg of Avicel PH101, were mixed together. Both the individual blends were sieved through 40 number mesh size. The two individual blends were blended for 30 minutes at 100 RPM. Final blend underwent direct compression in tablet punching machine. The hardness of the tablet was set to 10 kPa. The final weight of the core tablet is 150 mg. The coating solution was prepared using 80 mg of HPMC K100M. The coating was 6 wt %. The final coat was done with Eduragit L-100 & S-100 in IPA & water solution with 6-8 wt %.

Example 11 (NIP-BDS-15): 9 mg of Budesonide was mixed and coated with 40 mg guar gum. Further, 0.5 mg of Aerosil, 0.5 mg of magnesium stearate, and 40 mg of Avicel PH101, were mixed together. Both the individual blends were sieved through 40 number mesh size. The two individual blends were blended for 30 minutes at 100 RPM. Final blend underwent direct compression in tablet punching machine. The hardness of the tablet was set to 9 kPa. The final weight of the core tablet is 150 mg. The coating solution was prepared using 60 mg of HPMC K100M. The coating was 6 wt %.

Example 12 (NIP-BDS-16): 0.5 mg of Aerosil, 0.5 mg of magnesium stearate, and 20 mg of Avicel PH101 were mixed together with budesonide 9 mg. Individual blends were sieved through 40 number mesh size. The two individual blends were blended for 30 minutes at 100 RPM. Final blend underwent direct compression in tablet punching machine. The hardness of the tablet was set to 9 kPa. The final weight of the core tablet is 150 mg. The coating solution was prepared using 120 mg of HPMC K100M. The coating was 6 wt %.

The tablets based on Example 1 to 12 were then taken for further studies.

Table 1 below indicates the ingredients of the formulation according to Examples 1 to 12 along with hardness.

| | | | | | | | | | | | | | Time |
|---|---|---|---|---|---|---|---|---|---|---|---|---|---|
| | | | | | | | | | | | | | to |
| | | Guar | HPMC | HPMC | HPMC | HPMC | HPMC | MCC | Aerosil | Magnesium | Tablet | | release |
| | BDS | gum | E5 | K4M | K15M | K100LVCR | K100M | PH101 | 200 | Stearate | Weight | | 80% |
| Examples | (mg) | (mg) | (mg) | (mg) | (mg) | (mg) | (mg) | (mg) | (mg) | (mg) | (mg) | Hardness | drug |
| Example-1 | 9 | 50 | | | | | | 40 | 0.5 | 0.5 | 100 | 4.9 | 2 h |
| (BDS-NIP- | | | | | | | | | | | | | |
| 001) | | | | | | | | | | | | | |
| Example-2 | 9 | 50 | | | | | 50 | 40 | 0.5 | 0.5 | 150 | 9 | 2 h |
| (BDS-NIP- | | | | | | | | | | | | | |
| 002) | | | | | | | | | | | | | |
| Example-3 | 9 | 10 | 30 | | | | | 50 | 0.5 | 0.5 | 100 | 9 | 2 h |
| (BDS-NIP- | | | | | | | | | | | | | |
| 003) | | | | | | | | | | | | | |
| Example-4 | 9 | 25 | 30 | | | | | 35 | 0.5 | 0.5 | 100 | 9 | 4 h |
| (BDS-NIP- | | | | | | | | | | | | | |
| 004) | | | | | | | | | | | | | |
| Example-5 | 9 | 30 | | 20 | | | | 40 | 0.5 | 0.5 | 100 | 9 | ND* |
| (BDS-NIP- | | | | | | | | | | | | | |
| 005) | | | | | | | | | | | | | |
| Example-6 | 9 | 30 | | | 20 | | | 40 | 0.5 | 0.5 | 100 | 9 | ND* |
| (BDS-NIP- | | | | | | | | | | | | | |
| 006) | | | | | | | | | | | | | |
| Example-7 | 9 | 30 | | | | 20 | | 40 | 0.5 | 0.5 | 100 | 9 | ND* |
| (BDS-NIP- | | | | | | | | | | | | | |
| 007) | | | | | | | | | | | | | |
| Example-8 | 9 | 30 | | | | | 20 | 40 | 0.5 | 0.5 | 100 | 9 | ND* |
| (BDS-NIP- | | | | | | | | | | | | | |
| 008) | | | | | | | | | | | | | |
| Example-9 | 9 | — | | | | | 100 | 40 | 0.5 | 0.5 | 150 | 9 | ND* |
| (BDS-NIP- | | | | | | | | | | | | | |
| 013) | | | | | | | | | | | | | |
| Example- | 9 | 20 | | | | | 80 | 40 | 0.5 | 0.5 | 150 | 10 | ND* |
| 10 | | | | | | | | | | | | | |
| (BDS-NIP- | | | | | | | | | | | | | |
| 014) | | | | | | | | | | | | | |
| Example- | 9 | 40 | | | | | 60 | 40 | 0.5 | 0.5 | 150 | 9 | ND* |
| 11 | | | | | | | | | | | | | |
| (BDS-NIP- | | | | | | | | | | | | | |
| 015) | | | | | | | | | | | | | |
| Example- | 9 | — | | | | | 120 | 20 | 0.5 | 0.5 | 150 | 9 | ND* |
| 12 | | | | | | | | | | | | | |
| (BDS-NIP- | | | | | | | | | | | | | |
| 016) | | | | | | | | | | | | | |

*ND = The drug release was not detected at 24 hours in in-vitro dissolution mimicking large intestine media. However, 100% drug release was shown when Rat's large intestinal microbes were added to the same media confirmed by the experiments conducted.

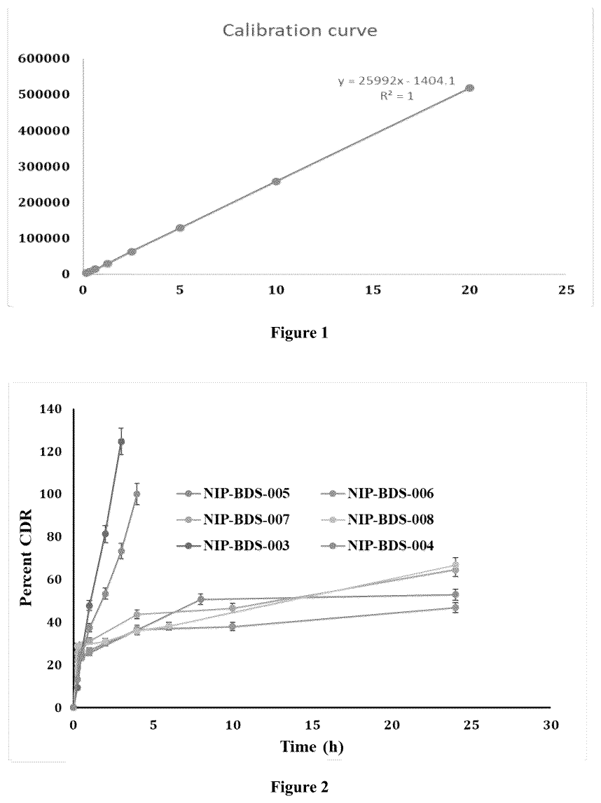

Method for detection of drug (Budesonide): HPLC was used to quantify the amount of drug released at different time points. An isocratic method comprising of 0.2:0.8 ACN:pH 4.5 ammonium acetate buffer in Chromasol C18 150 mm×5 μm column at flow rate of 1 mL/min. Retention time was found to be at 2.05 min with linearity between 156.25 ng/mL-20 μg/mL. FIG. 1 shows the HPLC calibration curve of Budesonide (BDS).

In vitro dissolution testing: Dissolution testing was performed in USP type II basket type apparatus at 37±2° C. in 1000 mL pH 6.8 phosphate buffer at 100 rpm to maintain sink condition. However, failure in maintenance of sink condition led to the use of 10% ethanol to maintain sink condition. Samples were taken at different time points and subjected to UV/HPLC analysis respectively.

### Results

FIG. 2 indicates the percent cumulative drug release with respect to time. From the results, it is clear that amount of HPMC K4M can be modified to attain greater than 80 percent drug release within 8-10 h.

Accordingly, guar gum was used as colon specific rate controlling polymer. At 50 mg, the tablet failed to release the drug in phosphate buffer pH 7.4 in the presence and absence of 50 mg of HPMC K100M (BDS-NIP-001 and BDS-NIP-002). HPMC K100M was used to increase the hardness from 4.9 (Without binder BDS-NIP-001) to an acceptable range i.e. 8-10 kg/sq. cm. and to improve water uptake followed by swelling and drug release. So lesser viscosity grades of HPMC were investigated as a binder and rate controlling polymer to give sustained drug release independent of tablet hardness up to 10 h by reducing the amount of Guar gum. When guar gum concentration was increased from 10 mg to 25 mg (BDS-NIP-003 and BDS-NIP-004) with HPMC E5 as binder, the release was sustained from 2 h to 4 h. Further, keeping guar gum constant at 30 mg, different grades of HPMC were screened to sustain the release profile till 10 h. BDS-NIP-005 containing HPMC K4M showed 50.71% drug release within 8 h while, BDS-NIP-006 containing HPMC K15M showed 37.94% drug release within 10 h. BDS-NIP-007 containing HPMC K100LVCR released 46.57% drug within 10 h whereas HPMC K100M (Only 20 mg compared to 50 mg in batch BDS-NIP-002) showed only 66.78% within 24 h.

Owing to the matrix holding capacity of HPMC K100M, it was varied in combination with guar gum to sustain the drug release and the dissolution study was performed in presence of 0.5 percent tween 80 in 1000 mL of pH 7.4 phosphate buffer.

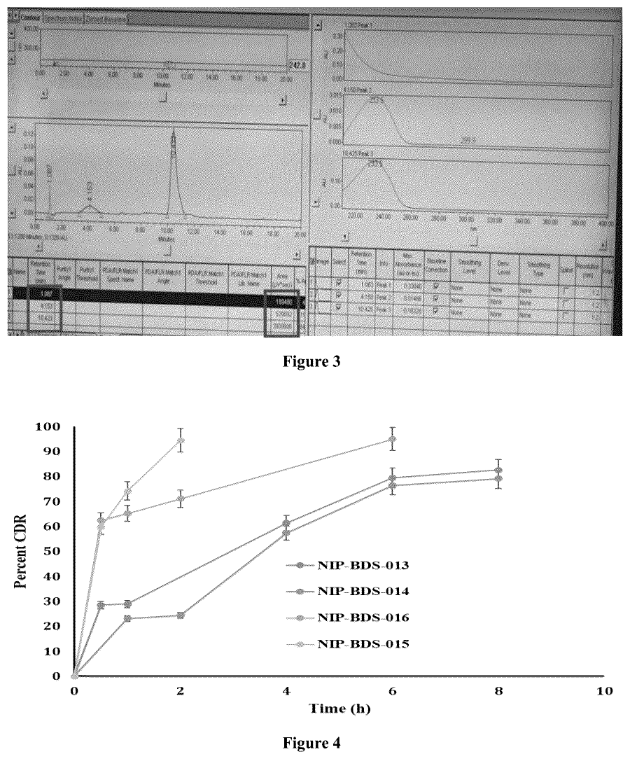

BDS interacted with tween 80 and resulted in unidentifiable peaks in HPLC as shown in the chromatogram in all samples (FIG. 3).

It was concluded that tween 80 could not be used to maintain sink condition in the dissolution media. Further, out of different co-solvents screened, 10 percent ethanol was found to maintain sink conditions in vitro by UV-vis spectroscopy. This was further added in subsequent dissolutions.

### Effect of Varying Guar Gum and HPMC K100M on Release Profile

TABLE 2
Drug release intervals

| | % Drug Released | | | |
|---|---|---|---|---|
| Time | Example-9 | Example-10 | Example-11 | Example-12 |
| (h) | (NIP-BDS-013) | (NIP-BDS-014) | (NIP-BDS-015) | (NIP-BDS-016) |
| 0.5 | 28.48 | — | 59.74 | 62.3 |
| 1 | 28.9 | 23.15 | 74.15 | 65.2 |
| 2 | — | 24.3 | 94.4 | 71 |
| 4 | 61.3 | 57.37 | — | 95.6 |
| 6 | 79.4 | 76.34 | — | — |
| 8 | 82.6 | 79.1 | — | — |

Drug release studies for the above batches indicating NIP-BDS-014 as lead batch for core optimization is also confirmed from the drug release studies by USP Type I apparatus in 10% ethanol in 1000 ml phosphate buffer pH 7.4 at 100 rpm (FIG. 4). This was also observed in the matrix integrity images after 8 h and 24 h (FIG. 5).

Eudragit coating: 20 BDS-NIP-014 tablets were taken with placebo tablets (10 mm punch diameter up to 170 g) and were coated with Eudragit L100 and S100 (1:1) in IPA:Water (90:10) containing 10% w/v TEC to achieve 13-15% weight gain.

TABLE 3
Enteric coating parameters optimized for NIP-BDS-014 core tablets
Ganson Inone Coating Parameters

| Inlet/Temp | 90%/55° C. |
|---|---|
| Outlet/Temp | 30%/28° C. |
| Feed rate | 3 |
| Pan rpm | 3-12 |
| Atomization Pressure | 22 |
| Fan pressure | 20 |
| Weight gain | 14.6% |

The tablets were subjected to disintegration testing for 2 h in 0.1N HCL to investigate the integrity of film coating. Due to sharp edges of the tablet, slight pores were observed, however, the film integrity was not compromised. The coated tablets were subjected to disintegration test in the presence of 0.1N HCl for 2 h. Absence of swelling indicated efficient coating of tablets compared to uncoated tablet. The batch was further subjected to dissolution studies in the presence and absence of rat cecal content.

Sequential dissolution study: Dissolution of uncoated and coated tablets and dissolution in presence and absence of rat cecal content 4% w/v was performed by subjecting the tablets to 1000 mL of 0.1N HCl for 1 h, 1000 mL of pH 6 phosphate buffer with 10% ethanol for 2.5 h and up to 30 h in pH 6.8 phosphate buffer with 10% ethanol at 37±2° C. and 100 rpm to maintain sink conditions. The samples were analysed by UV-vis spectroscopy at 243 nm and the results were confirmed by HPLC analysis. Further, sequential dissolution samples subjected to HPLC analysis demonstrated that coated tablets released about 19.16% drug within 30 h while uncoated tablets released 48.60% drug within 30 h with the absence of drug detection in 0.1N HCl owing to the absence of ethanol resulting in absence of sink condition. Matrix swelling behaviour as indicated in FIG. 5 showed slight swelling on the edges owing to pores on sharp edges for coated tablets. For uncoated tablets complete swelling was observed after 1 h and 30 h. Film coating prevented swelling and sustained the drug release.

Dissolution study in the presence of cecal content: For estimation of drug in cecal content, protein precipitation and extraction were performed by diluting samples aliquots 10× by ethanol and centrifuging for 30 min at 10000 rpm. The supernatant was then passed through 0.22μ filter and subjected to UV and HPLC analysis

Table 4 below demonstrates the results of dissolution of uncoated BDS-NIP-014 (prototype I) and coated BDS-NIP-014 (prototype II) in presence and absence of cecal content respectively.

| Time interval | Prototype-I | Prototype-II | Prototype-II with |
|---|---|---|---|
| (h) | Uncoated | Coated | Cecal content |
| 0 | 0 | 0 | 0 |
| 0.5 | 0 | 0 | 0 |
| 1 | 0 | 0 | 0 |
| 2.5 | 2.8 | 0 | 0 |
| 4 | 4.2 | 0 | 0 |
| 6 | 6.9 | 2 | 0 |
| 8 | 11.3 | 4.1 | 0 |
| 10 | 16.6 | 7.1 | 0 |
| 24 | 31 | 12.6 | 100 |
| 30 | 48.6 | 19.1 | 100 |

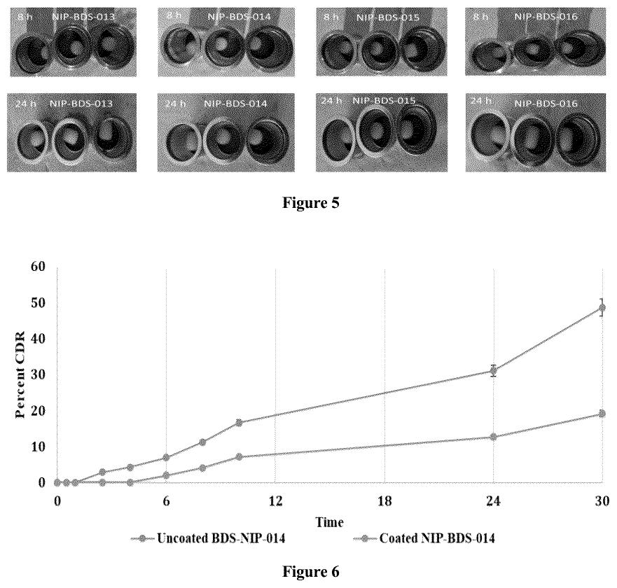

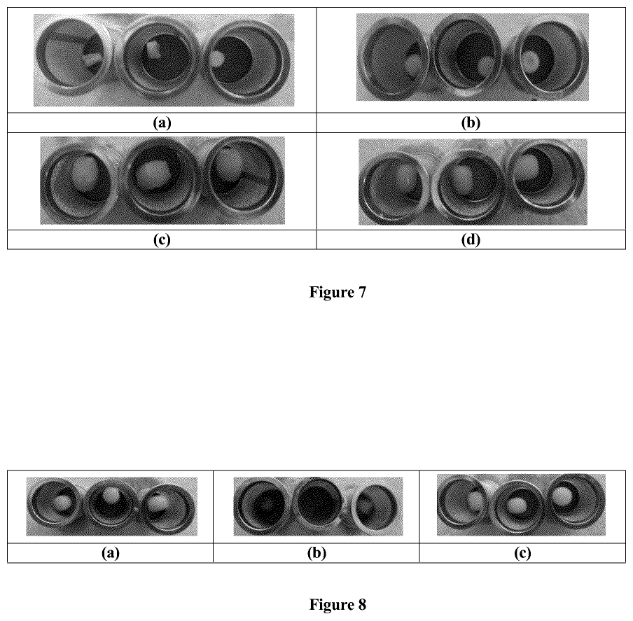

Drug release studies for the above uncoated BDS-NIP-014 (prototype I) and coated BDS-NIP-014 (prototype II) were further evaluated for sequential dissolution by HPLC under the following conditions—1 h in 0.1N HCL, 2.5 h in pH 6 phosphate buffer with 10% ethanol and up to 30 h in pH 6.8 phosphate buffer with 10% ethanol. The results are shown in terms of complete drug release in FIG. 6 and matrix integrity in FIG. 7. Further, the matrix integrity after sequential dissolution with 4% cecal content and without cecal content has also been shown in FIG. 8.

In conclusion, based on the tableting & in-vitro dissolution methodology discussed above it was found that 98% (including 2% of a possible mathematical error) of the Budesonide was released in the large intestine media.

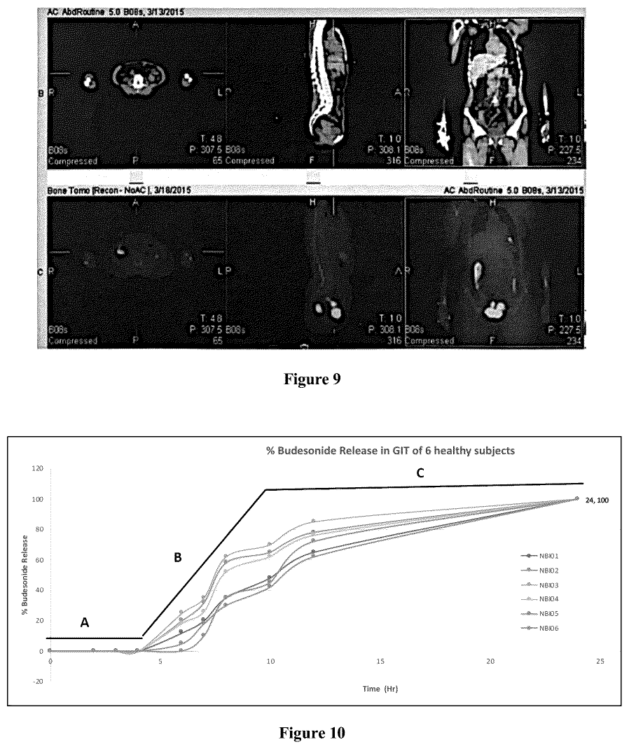

Scintigraphy study: As final conclusive evidence, a 24 hours scintigraphy study was conducted with six healthy human subjects to check the release pattern of the tablet (Budesonide) in GI tract of six human subjects for 24 hours. The formulation was radiolabeled with 99M-Tc-Pertechnatet for the measurement of drug under the gamma camera. The radiolabeled formulation was passed through the quality control checks in order to confirm the radiolabeling efficiency in vitro as well as in-vivo. All subjects were passed through the 10 different dynamic & static imaging up to 24 hours starting from 0 hr. The SPECT imaging along with gamma camera acquired together to check the accurate location of drug post 24 hours (FIG. 9).

Ethical considerations: This study was conducted in compliance with ICH GCP, ICMR, GCLP, Drugs and Cosmetics Act, 1940, New Drugs and Clinical Trials Rules—2019 [Gazette Notification G.S.R.227 (E), Dated 19 Mar. 2019]. The Ethics approval was obtained by an independent ethics committee “Society for Research & Welfare (SRW) with approval number of SRW-IEC/NR/2022/CL/003.

It was observed from the scintigraphy study that there was 0% drug release found in upper part of GI tract such as esophageal tract, stomach and small intestine. Esophageal transit time (ETT) for all participants was less than 5 seconds and it was captured in the first dynamic images. Further, the gastric emptying time of formulation was found within 1 hour for all of the subjects. For subjects 1, 2, 3, 4, and 5, the tablet reached the ileococcal junction (initial part of the colon) by the 8 hr mark and for subject 6, the tablet reached the ileococcal junction by the 9 hr mark.

The radiolabeled test tablet stays in the stomach as a single entity without any release or leakage of radioactivity from a tablet in the first 2 hours. The disintegration of the tablet was started once it reaches to intestinal part or say in between 2.67±1.63-hours. In most of cases tablet reach intestine around 2 hours and only in one case it reaches at 6 hours, it reaches the intestine early as well.

At 2-6 h imaging, the radioactivity was observed at the gastric cavity as a single entity, and some light spots at the stomach also appeared in the intestine, which indicate the intestinal arrival time. Hence total gastric residence time was somewhere 2.67±1.67 hours and intestine arrival time was 2.67 hours.

At 20-24 h images, radioactive formulation reached to the ileocecal junction and enter in ascending transverse colon and in some 24 hours images (a total of 6 subjects) shows complete disintegration of tablets i.e., 85% to 100%.

FIG. 10 demonstrates the release pattern of drug in the GI tract. (A)—The drug in the stomach & small intestine and tablet remains intact without drug release (B)—Drug showed release at the ileocaecal junction/arrival of colon and drug release started (C)—Drug distributed in the colon and complete disintegration of tablet monitor.

The Gamma imaging was done at different time till 24 hours in order to confirm the exact location of formulation and its release through GI tract of all human subjects and there was no release (0% drug) of activity or drug at the intestine, which indicated that the tablet is able to deliver the drug more than 99% at the colon. There was no release of drug from the tablets observed in the stomach in all subjects. It indicated that the formulation does not interact with the gastric juice and is passed to the small intestine as a whole intact without disintegration.

Conclusively, the formulation reached successfully the colon & ileocaecal junction without the release of the drug in the upper part of GI tract such as the stomach and small intestine. The formulation delivers the active ingredient or the drug to the predetermined location in the GI tract with more than 90%, preferably more than 95%, and most preferably more than 99% success.

The foregoing description of the invention has been set merely to illustrate the invention and is not intended to be limiting. Since the modifications of the disclosed embodiments incorporating the spirit and substance of the invention may occur to the person skilled in the art, the invention should be construed to include everything within the scope of the disclosure.

1. An oral drug delivery formulation comprising:
a central core;
an inner coating of the central core; and
an optional outer enteric coating
wherein the central core comprises an active ingredient or drug having a primary coating based on plant extract or their derivatives and one or more disintegration agent along with one or more pharmaceutically acceptable excipient.

2. The formulation as claimed in claim 1, wherein the plant extract or their derivatives is selected from galactomannan, polysaccharide, chitosan, gum derived from plants.

3. The formulation as claimed in claim 1, wherein plant extract or their derivatives are obtained from Trigonella foenum-graecum, Cyamopsis tetragonoloba, Astragalus gummifer, Acasia senegal, Acacia nilotic.

4. The formulation as claimed in claim 1, wherein the plant extract or a derivative thereof is based on guar gum.

5. The formulation as claimed in claim 1, wherein the disintegration agent is selected from a micro crystalline cellulose, cross linked PVP, cellulose, PVP, starch, alginic acid and calcium silicates.

6. The formulation as claimed in claim 1, wherein the inner coating includes at least one polymer derived from acrylic acid, alginates, polysaccharides, and cellulose, preferably selected from a methacrylic acid polymer or pullulan.

7. The formulation as claimed in claim 1, wherein the inner coating comprises hydroxypropyl methylcellulose (HPMC).

8. The formulation as claimed in claim 1, wherein the inner coating further comprises at least one plasticizer, preferably selected from diethyl phthalate triacetin.

9. The formulation as claimed in claim 1, wherein the active ingredient or drug is selected from Budesonide.

10. The formulation as claimed in claim 1, wherein the outer enteric coating is selected from at least one of semisynthetic polymer, anionic copolymer, plant extracts and plant derived resins and gum.

11. The formulation as claimed in claim 1, wherein the outer enteric coating comprises an anionic copolymer selected from Eudragit-L-100 or Eudragit S-100 or a combination thereof.

12. The formulation as claimed in claim 1, wherein the amount of outer enteric coating is in the range of 1 to 50 wt %, primary coating is in the range of 1 to 25 wt %, excipient of the formulation is in the range of 1 to 50 wt %, disintegration agent of the formulation is in the range of 1 to 30 wt % and plant extract or their derivatives is in the range of 1 to 50 wt % based on the total weight of the formulation.

13. A method for preparing an oral drug delivery formulation claimed in claim 1 comprising the following steps:
primary coating of active ingredient or drug powder followed by drying and sifting to obtain a first mix;
granulating the first mix to obtain semi-dried granules;
milling the semi-dried granules followed by sifting to obtain sift milled granules;
predetermined amounts of disintegration agent along with one or more pharmaceutically acceptable excipients are mixed together and sieved to obtain a second mix; blending the sift milled granules and the second mix to obtain the central core of formulation;
coating the central core to obtain the oral drug delivery formulation.

14. The method as claimed in claim 13, wherein the coating includes at least one polymer derived from acrylic acid, alginates, polysaccharides, and cellulose, preferably selected from a methacrylic acid polymer or pullulan, preferably comprises hydroxypropyl methylcellulose (HPMC).

15. The method as claimed in claim 13, wherein the blending is followed by a direct compression in tablet punching machine to obtain a tablet.

16. The method as claimed in claim 13, wherein the tablet is outer enteric coated, preferably comprising Eudragit-L-100 or Eudragit S-100 or a combination thereof.

17. The method as claimed in claim 13, wherein the sifting is carried out in sieves having mesh size in the range of 30 to 400.

18. The method as claimed in claim 12, wherein the method comprises at least one step of wet granulation, direct compression, compressed coating and wet coating.

19. The method as claimed in claim 12, wherein the drying is carried out at a temperature range of 25 to 65° C.

20. A method for the treatment of a patient by delivering a drug to a predetermined location in the gastrointestinal tract, comprising administering the oral drug delivery formulation as claimed in claim 1 in a patient in need of the said drug.

21. The method as claimed in claim 16, wherein the predetermined location in the gastrointestinal tract is colon.

22. The method as claimed in claim 16, wherein the drug is Budesonide.

23. Use of the oral drug delivery formulation as claimed in claim 1 for delivering a drug to a predetermined location in the gastrointestinal tract, preferably colon.
